(19) United States
(12) Patent Application Publication
(10) Pub. No.: US 2025/0072988 A1
(43) Pub. Date: Mar. 6, 2025

(54) MINIATURE DEVICE AND SYSTEM FOR REMOTELY CONTROLLING IT WITHIN A PATIENT

(71) Applicant: BIONAUT LABS LTD., Herzliya (IL)

(72) Inventors: Michael SHUSTOV, Haifa (IL); Aviv SHTEIN, Tel Aviv (IL); Michael SHPIGELMACHER, Los Angeles, CA (US); Alex KISELYOV, San Diego, CA (US); Be'eri Berl KATZNELSON, Kiryat Tivon (IL); Michael KARDOSH, Kiryat Ono (IL); Florent CROS, Los Angeles, CA (US)

(73) Assignee: BIONAUT LABS LTD., Herzliya (IL)

(21) Appl. No.: 18/789,591

(22) Filed: Jul. 30, 2024

Related U.S. Application Data
(60) Provisional application No. 63/517,457, filed on Aug. 3, 2023.

(51) Int. Cl. A61B 34/00; A61B 90/00

(52) U.S. Cl. CPC A61B 34/73; A61B 90/39; A61B 2034/731; A61B 2090/3954

(57) ABSTRACT

A miniature device is provided for facilitating performing a therapeutic activity at a target site in a patient. The miniature device comprises a body defining a longitudinal axis extending along a longitudinal direction, and is configured to be maneuvered to move in the longitudinal direction by a linear magnetic field aligned along the longitudinal axis, and to be rotated about the longitudinal axis by a rotating magnetic field rotating about the longitudinal axis.

### TECHNOLOGICAL FIELD

The presently disclosed subject matter relates to systems and miniature devices configured to navigate within a patient to perform a therapeutic activity at a predetermined target site therewithin, and in particular to such systems which use magnetic fields to direct operation of miniature devices within a patient.

### BACKGROUND

Remote control of miniature medical devices inside the human body can be useful for a variety of purposes, including, but not limited to, delivery of therapeutic payloads, diagnostics, and surgical procedures. Such devices may include microscale or nanoscale robots, medical tools, “smart pills,” etc. The most suitable type of motion, e.g., forward, screwing, etc., may depend on the environment, e.g., including the type of matter, in which the miniature device is situated.

### SUMMARY

According to an aspect of the presently disclosed subject matter there is provided a miniature device for facilitating performing a therapeutic activity in a patient, the miniature device comprising a body defining a longitudinal axis extending along a longitudinal direction, the miniature device being configured to be maneuvered to move in the longitudinal direction by a linear magnetic field, and to be rotated about the longitudinal axis by a rotating magnetic field.

It will be appreciated that herein the specification and appended claims, the term “facilitating performing a therapeutic activity” may include, but is not limited to, executing the therapeutic activity, delivering and/or activating a medicament, prodrug, and/or component thereof, performing or facilitating the performance of imaging, recording, monitoring, and/or measurement, and/or performing or facilitating the performance of a diagnostic activity.

It will be further appreciated that the term “longitudinal,” e.g., when used in connection with the longitudinal axis, is not meant to be limiting, and is used herein the specification and appended claims as being descriptive of typical, but non-limiting, embodiments, in which the body of the miniature device extends generally along the longitudinal axis. However, one having skill in the art will recognize that a miniature device may be provided in which the body does not extend along the longitudinal axis/direction (e.g., a general spherical shape) or extends along an axis/direction other than the longitudinal; in such cases, descriptions and/or definitions of components expressed herein with reference to the longitudinal axis/direction are not to be understood with reference to the axis along which the body extend.

It will be further appreciated that the term “linear” when used in reference to a magnetic field is to be understood in a geometric sense, i.e., that the local magnetic field in the area of interest (in the above description this may be the body of the miniature device or a relevant portion thereof, such as the steering magnet described below) is substantially linear, i.e., the force vectors are substantially aligned in the linear direction. It is not to be understood as referring to the uniformity of the magnetic field.

The linear magnetic field may be substantially aligned along the longitudinal axis, and the rotating magnetic field may rotate substantially about the longitudinal axis.

It will be appreciated that herein the specification and appended claims, the term “substantially,” in particular when used with reference to axes and/or the orientation of fields relative to one or more axes, may be understood as imparting a wide scope to the term modified thereby. One having skill in the art will recognize that the application in which the miniature device is to be used may allow for a high tolerance. For example, if the miniature device is maneuvered through a tight space, an applied linear magnetic field may not need to be aligned closely with the longitudinal axis of the body. Moreover, in some situations is may be desirable for the magnetic field to deviate from the orientation defined by the longitudinal axis, for example to moderate the component of force therealong/thereabout.

The miniature device may be configured to be driven forward by the rotation about the longitudinal axis.

The miniature device may comprise a steering magnet defining a pole axis spanning between its poles, the steering magnet being pivotable about a pivot axis between a first position in which the pole axis is substantially parallel to the longitudinal axis, and a second position in which the pole axis is substantially perpendicular to the longitudinal axis.

The pivot axis may be substantially perpendicular to the longitudinal axis and

substantially perpendicular to the pole axis.

The miniature device may comprise an axle along the pivot axis, the steering magnet being mounted on the axle to facilitate the pivoting.

The body may comprise a cavity housing the steering magnet, the cavity having a substantially circular cross-section in a plane perpendicular to the pivot axis.

The miniature device may further comprise a pivot-limiting arrangement configured to constrain the range of motion of the steering magnet between its first and second positions.

The pivot-limiting arrangement may comprise:
a protrusion projecting from the magnet; and
two stoppers, each being fixedly disposed with the body so as to obstruct the protrusion when the steering magnet reaches one of its first and second positions.

The body may be formed with a channel spanning between the two stoppers, the channel being configured to accommodate the protrusion therein when the steering magnet pivots between its first and second positions.

The pivot-limiting arrangement may be configured to selectively arrest the protrusion in its first and/or second position.

The steering magnet may have a substantially spherical shape.

The body may comprise an exterior thread having an axis substantially coincident with the longitudinal axis.

At least one end of the body along the longitudinal axis may be formed as a wedge.

The miniature device may comprise at least one radiopaque fiducial. The at least one radiopaque fiducial may be eccentrically located relative to the longitudinal axis. The at least one radiopaque fiducial may be magnetic.

The linear magnetic field may comprise a magnetic gradient along the longitudinal axis.

According to another aspect of the presently disclosed subject matter, there is provided a driving arrangement configured to selectively generate one of a linear magnetic field and a rotating magnetic field (i.e., it is configured to generate both, and to selectively generate one of a linear and a rotating magnetic field), thereby remotely directing motion of a miniature device as described above.

According to another aspect of the presently disclosed subject matter, there is provided a system configured to facilitate performing a therapeutic activity in a patient, the system comprising:
a miniature device as described above; and
a driving arrangement as described above.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a perspective view of an example of a miniature device according to the presently disclosed subject matter;

FIGS. 2A and 2B are cross-sectional views of the miniature device, taken along line II-II in FIG. 1, with a steering magnet thereof in first and second positions, respectively; and

FIG. 3 is a transparent perspective view of the miniature device illustrated in FIG. 1, with the steering magnet in its first position.

### DETAILED DESCRIPTION

In the following detailed description, numerous specific details are set forth in order to provide a thorough understanding of the presently disclosed subject matter. However, it will be understood by those skilled in the art that the presently disclosed subject matter may be practiced without these specific details. In other instances, well-known methods, procedures, and components have not been described in detail so as not to obscure the presently disclosed subject matter.

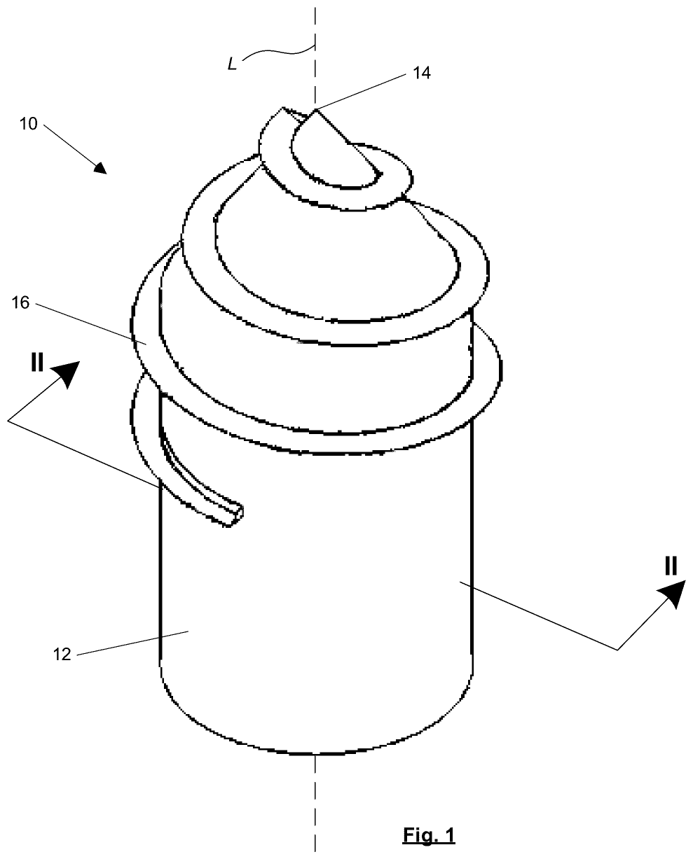

As illustrated in FIG. 1, there is provided a miniature device, which is generally indicated at 10, for facilitating performing a therapeutic activity at a target site in a patient. According to some examples, the therapeutic activity comprises delivery of one or more chemical compounds of medicinal, diagnostic, evaluative, and/or therapeutic relevance, one or more small molecules, biologics, cells, one or more radioisotopes, one or more vaccines, etc. (hereinafter “therapeutic component”), for example via body fluids, an anatomic lumen, and/or soft tissue to the target site. According to some examples, the target site is in the patient's brain and/or other areas of the central nervous system.

According to other examples, the therapeutic activity comprises performing a cutting operation at the target site.

It will be appreciated that for the sake of the present description, the term “target site” may be used herein the present description and appended claims to refer to the location where the system 10 acts, even if the location which is treated is located elsewhere. For example, the system 10 may be configured to perform a cutting operation at a location in order to access an adjacent location to deliver a therapeutic component thereto. In such a case, the location which undergoes the cutting operation may be referred to herein as the “target site,” even though it is not the ultimate recipient of the therapeutic component. In some cases, both the location at which the cutting operation is performed, as well as the location which is the ultimate recipient of the therapeutic component, may each be described as a “target site,” for example interchangeably within a single description of a therapeutic activity; in such cases, one having ordinary skill in the art will appreciate from context which location is referred to by each use of the term “target site.”

The miniature device 10 is configured to be remotely controlled by an externally applied magnetic field having parameters which are customized to cause the miniature device to undergo a predetermined motion (e.g., forward motion, rotational motion, etc.). The parameters may include, but are not limited to, strength, direction, rate of variation, etc. In order for the miniature device 10 to perform complex motions, e.g., traveling along a non-linear path, etc., the parameters may be continuously adjusted, e.g., according to a pre-determined program, based on feedback received regarding the location of the miniature device 10, a combination of the above, etc.

The miniature device 10 comprises an elongate body 12 defining a longitudinal axis L which extends in a longitudinal direction. As will be described below, the miniature device 10 is configured to operate in a linear mode, in which a linear magnetic field operates to move it in the longitudinal direction, and a rotating mode, in which rotating magnetic field operates to rotate it about the longitudinal axis L.

According to some examples, a top end of the body along the longitudinal axis L may be formed as a wedge, for example as a cylindrical pyramid. The wedge may define a point 14 at its distal end. According to some examples, the top side of the body may be elongated, for example forming a needle, and having a distal point.

According to some examples, the miniature device 10 is configured to move in the longitudinal direction in the rotating mode. Accordingly, the body 12 may comprise an exterior thread 16 formed about an axis substantially coincident with the longitudinal axis. In this way, the exterior thread 16 converts the torque of the rotating body 12 to linear motion along the longitudinal axis. This may be useful, e.g., when the miniature device 10 is traveling through a high-resistance environment, such as a viscous fluid, non-liquid tissue, etc.

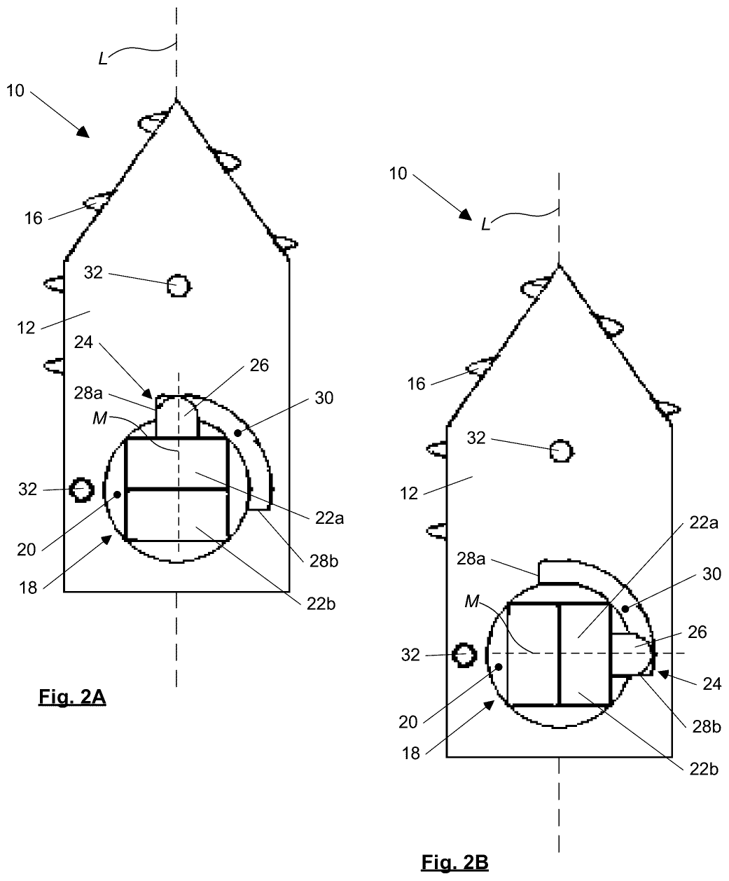

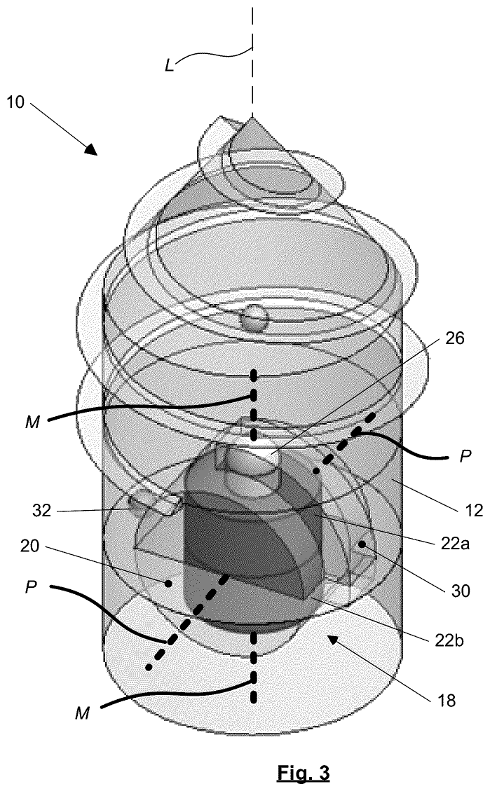

As illustrated in FIGS. 2A through 3, the miniature device 10 comprises a steering magnet 18, which is configured to be acted on by the externally applied magnetic field to effect a predetermined motion of the miniature device. In particular, it may be moveable between two different positions, each corresponding to one of the modes of the miniature device 10. The steering magnet may have any suitable shape, including, but not limited to, cylindrical, spherical, etc.

The body 12 of the miniature device 10 may comprise a cavity 20, within which the steering magnet 18 is accommodated. The cavity 20 is configured to permit motion of the steering magnet 18 between its first and second positions. According to some examples, the cavity 20 has a substantially circular cross-section.

The steering magnet 18 comprises north and south poles 22a, 22b, disposed on opposite ends of a pole axis M spanning therebetween. In the present disclosure, different elements indicated by a single reference numeral and distinguished by their trailing letters may be collectively indicated using the single reference numeral without a trailing letter, e.g., reference numeral 22 may be used to collectively indicate poles 22a and 22b.

The steering magnet 18 is configured to pivot between a first position (illustrated in FIG. 2A) and a second position (illustrated in FIG. 2B). In the first position of the steering magnet 18, which corresponds to the linear mode of the miniature device 10, the pole axis M of the steering magnet is substantially parallel to (including coincident with) the longitudinal axis L of the miniature device, i.e., the north and south poles 22a, 22b are aligned with one another along the longitudinal axis L. In the second position of the steering magnet 18, which corresponds to the rotating mode of the miniature device 10, the pole axis M of the steering magnet is substantially perpendicular to the longitudinal axis L of the miniature device, i.e., the north and south poles 22a, 22b are aligned with one another transverse to the longitudinal axis L.

The steering magnet 18 is configured to pivot about a pivot axis P (shown in FIG. 3) between its first and second positions. According to some examples, the pivot axis P is substantially perpendicular to the longitudinal axis L and substantially perpendicular to the pole axis M. It will be appreciated that while all three axes (i.e., the pivot, longitudinal, and pole axes) may sometimes be mutually perpendicular to one another, for example when the steering magnet 18 is in its second position corresponding to the rotating mode of the miniature device 10, this is not always the case, e.g., when the steering magnet is in its first position corresponding to the linear mode of the miniature device, longitudinal and pole axes are parallel or coincident with each other.

In the linear mode of the miniature device 10, i.e., when the steering magnet 18 is in its first position, an externally applied linear magnetic field, aligned along the longitudinal axis L, causes the miniature device to move in a path along the longitudinal axis. According to some examples, the externally applied linear magnetic field has a gradient in the direction of the longitudinal axis L of the miniature device.

In the rotating mode of the miniature device, i.e., when the steering magnet 18 is in its second position, an externally applied rotating magnetic field, produced radially about the longitudinal axis Z (transverse to the pivot axis P and to the pole axis M of the steering magnet when in the second position), causes the miniature device to rotate about the longitudinal axis.

According to some examples, the steering magnet is mounted to an axle (not illustrated), aligned along the pivot axis P. The steering magnet 18 is configured to pivot on the axle.

The miniature device 10 may be formed with a pivot-limiting arrangement, which is generally indicated at 24, provided to facilitate proper operation of the steering magnet 18 to effect a predetermined motion of the miniature device in response to an externally applied magnetic field. The pivot-limiting arrangement is configured, inter alia, to constrain the range of motion, i.e., pivoting motion, of the steering magnet between its first and second positions.

The pivot-limiting arrangement 24 comprises a protrusion 26 projecting from the steering magnet 18. According to some examples, e.g., as illustrated in FIGS. 2A and 2B, the protrusion 26 may project from the steering magnet 18 generally in a direction along the pivot axis P. According to other examples (not illustrated), the protrusion 26 may project from the steering magnet 18 in any suitable direction, including, but not limited to, perpendicular to the pivot axis P. The pivot limiting arrangement 24 further comprises first and second stoppers 28a, 28b, associated, respectively, with the first and second positions of the steering magnet 18. Each of the stoppers 28 is configured to obstruct the protrusion 26, thereby preventing the steering magnet 18 from further pivoting.

According to some examples, the body 12 may be formed with a channel 30 to accommodate the protrusion 26 as it moves with the steering magnet 18, when the steering magnet pivots between its first and second positions. Accordingly, the channel 30 may be formed as an extension of the cavity 18. The channel 30 may span between the stoppers 28, wherein the ends of the channel constitute the stoppers. It will be appreciated that while the cavity 20 and channel 30 are illustrated in FIGS. 2A and 2B as being separated by a line, in practice the cavity 20 and channel 30 may constitute different portions of a single void formed within the body 12.

The steering magnet 18 may be pivoted between its two positions by any suitable method. According to some examples, a pivoting rotating magnetic field, which is produced radially about the pivot axis P, may be selectively applied to pivot the steering magnet to a desired position. As the pivot axis P is substantially perpendicular to the longitudinal axis L, the pivoting rotating magnetic field may be substantially orthogonal to the rotating magnetic field which is applied in the rotating mode, causing the miniature device 10 to rotate about the longitudinal axis L, and thus may be produced such that it does not interfere therewith. According to some examples, the pivoting rotating magnetic field may be maintained continuously while the miniature device 10 is controlled by the externally applied magnetic field, or for portions thereof, for example while ensuring that it is always produced radially about the pivot axis P, so as not to interfere therewith.

The miniature device 10 may comprise an arresting arrangement (not illustrated) configured to selectively arrest the steering magnet 18 in its first and/or second position. According to some examples, the arresting arrangement constitutes part of the pivot-limiting arrangement 24, e.g., the arresting arrangement may be configured to selectively arrest the protrusion 26 when the steering magnet 18 is in its first and/or second positions, thereby arresting the steering magnet. Accordingly, the steering magnet 18 may be secured in its first and second positions without requiring any external forces to maintain it in that position.

According to some examples of the arresting arrangement, the protrusion 26 comprises a latch configured to be received within a corresponding one or more bores formed in the body 12, each located such that when the latch is received therein, the steering magnet 18 is arrested at the first or second position. Alternatively, the body 12 may comprise one or more latches, each configured to be received within a bore formed in the protrusion, and each location such that when it is received within the bore, the steering magnet 18 is arrested at the first or second position. The latch may be controlled externally, for example by an externally applied magnetic field, electronic signal, electroacoustic, e.g., ultrasound, signal, etc., for example as is known in the art.

According to some examples of the arresting arrangement, portions of the channel 30 may be formed having L-shape, i.e., having legs projecting at an angle from the main portion of the channel which spans between the stoppers 28. The arresting arrangement is operated by maneuvering the steering magnet 18 such that the protrusion 26 enters one of the legs, thereby securing the steering magnet there in its first and/or second position. It will be appreciated that while the steering magnet 18 may not be strictly “secured” in a position by maneuvering the protrusion into one of the legs, herein the specification and appended claims, the term “secure” may be used in this context to refer to the leg being designed such that under normal operating conditions, the protrusion 26 will not be dislodged therefrom without being maneuvered therefrom by a user.

According to some examples, the miniature device 10 comprises one or more radiopaque fiducials 32, e.g., embedded in the body 12. The fiducials may be used during operation to assist a user in visualizing the position of the miniature device 10. At least one of the radiopaque fiducials 32 may be positioned eccentrically relative to the longitudinal axis L, thereby facilitating visualization of rotation of the miniature device.

According to some examples (not illustrated), the miniature device 10 may comprise a single radiopaque fiducial 32 positioned eccentrically relative to the longitudinal axis L, for example above the steering magnet 18 (i.e., wherein the up-down direction is along the longitudinal axis).

According to some examples, the one or more radiopaque fiducials 32 may be magnetic. This may be useful, e.g., to facilitate maneuvering the miniature device 10, for example by application of a targeted magnetic field.

According to some examples, the miniature device 10 as described above with reference to and as illustrated in FIGS. 1 through 2B may be part of a system, which further comprises a driving arrangement configured to selectively generate a linear magnetic field or a rotating magnetic field, thereby remotely directing motion of the miniature device. The driving arrangement may further comprise a controller configured to determine how to adjust the generated magnetic field so that a predetermined motion of the miniature device 10 is effected.

As alluded to above, the miniature device 10 may be operated in linear mode or rotating mode depending, inter alia, on the medium through which it is traveling or needs to pass. For example, when traveling through a low-resistance material, e.g., one having with a relatively low viscosity, the miniature device may be operated in linear mode, and when traveling through a high-resistance material, e.g., one having with a relatively high viscosity, a non-liquid tissue, etc., the miniature device may be operated in rotating mode, and be propelled linearly along its longitudinal axis L by the exterior thread 16, as described above. Accordingly, the miniature device 10 may traverse a path which passes through mediums of different resistances, being switched between its linear and rotating modes as necessary.

In addition, the miniature device 10 may be operated in linear mode to facilitate passing through a membrane, for example the pia mater, arachnoid mater, etc., or other similar tissue. The miniature device 10 may be operated to repeatedly tap the membrane with its point 14, thereby piercing the membrane, and facilitating passage therethrough.

It will be recognized that examples, embodiments, modifications, options, etc., described herein are to be construed as inclusive and non-limiting, i.e., two or more examples, etc., described separately herein are not to be construed as being mutually exclusive of one another or in any other way limiting, unless such is explicitly stated and/or is otherwise clear. Those skilled in the art to which this invention pertains will readily appreciate that numerous changes, variations, and modifications can be made without departing from the scope of the presently disclosed subject matter, mutatis mutandis.

1. A miniature device for facilitating performing a therapeutic activity in a patient, the miniature device comprising a body defining a longitudinal axis extending along a longitudinal direction, the miniature device being configured to be maneuvered to move in the longitudinal direction by a linear magnetic field, and to be rotated about the longitudinal axis by a rotating magnetic field.

2. The miniature device according to claim 1, wherein the linear magnetic field is substantially aligned along the longitudinal axis, and the rotating magnetic field rotates substantially about the longitudinal axis.

3. The miniature device according to claim 1, being configured to be driven forward by the rotation about the longitudinal axis.

4. The miniature device according to claim 1, comprising a steering magnet defining a pole axis spanning between its poles, the steering magnet being pivotable about a pivot axis between a first position in which the pole axis is substantially parallel to the longitudinal axis, and a second position in which the pole axis is substantially perpendicular to the longitudinal axis.

5. The miniature device according to claim 4, wherein the pivot axis is substantially perpendicular to the longitudinal axis and substantially perpendicular to the pole axis.

6. The miniature device according to claim 4, comprising an axle along the pivot axis, the steering magnet being mounted on the axle to facilitate the pivoting.

7. The miniature device according to claim 4, the body comprising a cavity housing the steering magnet, the cavity having a substantially circular cross-section in a plane perpendicular to the pivot axis.

8. The miniature device according to claim 4, further comprising a pivot-limiting arrangement configured to constrain the range of motion of the steering magnet between its first and second positions.

9. The miniature device according to claim 8, the pivot-limiting arrangement comprising:
a protrusion projecting from the magnet; and
two stoppers, each being fixedly disposed with the body so as to obstruct the protrusion when the steering magnet reaches one of its first and second positions.

10. The miniature device according to claim 9, the body being formed with a channel spanning between the two stoppers, the channel being configured to accommodate the protrusion therein when the steering magnet pivots between its first and second positions.

11. The miniature device according to claim 9, the pivot-limiting arrangement being configured to selectively arrest the protrusion in its first and/or second position.

12. The miniature device according to claim 4, the steering magnet having a substantially spherical shape.

13. The miniature device according to claim 1, wherein the body comprises an exterior thread having an axis substantially coincident with the longitudinal axis.

14. The miniature device according to claim 1, wherein at least one end of the body along the longitudinal axis is formed as a wedge.

15. The miniature device according to claim 1, comprising at least one radiopaque fiducial.

16. The miniature device according to claim 15, wherein the at least one radiopaque fiducial is eccentrically located relative to the longitudinal axis.

17. The miniature device according to claim 15, wherein the at least one radiopaque fiducial is magnetic.

18. The miniature device according to claim 1, wherein the linear magnetic field comprises a magnetic gradient along the longitudinal axis.

19. A driving arrangement configured to selectively generate one of a linear magnetic field and a rotating magnetic field, thereby remotely directing motion of a miniature device according to claims.

20. A system configured to facilitate performing a therapeutic activity in a patient, the system comprising:
a miniature device according to claim 1; and
a driving arrangement configured to selectively generate one of a linear magnetic field and a rotating magnetic field, thereby remotely directing motion of the miniature device.
